(19) United States
(12) Patent Application Publication
(10) Pub. No.: US 2023/0250489 A1
(43) Pub. Date: Aug. 10, 2023

(54) BIOMARKER, KIT, AND METHOD FOR DETECTION OR AUXILIARY DETECTION OF PROTON IRRADIATION AND USE THEREOF

(71) Applicant: Academy of Military Medical Sciences, Beijing (CN)

(72) Inventors: Hua GUAN, Beijing (CN); Shanshan Gao, Beijing (CN); Pingkun Zhou, Beijing (CN); Chenjun Bai, Beijing (CN); Yuchen Li, Beijing (CN); Hongling Zhao, Beijing (CN); Wen Zhang, Beijing (CN); Xiaochang Liu, Beijing (CN)

(73) Assignee: Academy of Military Medical Sciences, Beijing (CN)

(21) Appl. No.: 18/068,696

(22) Filed: Dec. 20, 2022

(30) Foreign Application Priority Data
Dec. 22, 2021 (CN) 202111575146.5

(51) Int. Cl. C12Q 1/689

(52) U.S. Cl. CPC C12Q 1/689

(57) ABSTRACT

The present disclosure relates to a biomarker, a kit, and a method for detection or auxiliary detection of proton irradiation and use thereof, and belongs to the field of biotechnology. The present disclosure provides a microbial biomarker for detection or auxiliary detection of proton irradiation, in which the microbiota includes at least one of Cyanobacteria, Epsilonbacteraeota, Anaerovorax and Helicobacter. In the present disclosure, whether a subject to be tested is exposed to proton radiation is accurately determined by detection of an abundance or relative abundance of DNA of the microbial biomarker. This is conductive to timely accident rescue, casualty treatment and clinical treatment application, which is of great significance to radiation treatment.

### CROSS-REFERENCE TO RELATED APPLICATION

This patent application claims the benefit and priority of Chinese Patent Application No. 202111575146.5, entitled “Biomarker, Kit, and Method for Detection or Auxiliary Detection of Proton Irradiation and Use Thereof” filed on Dec. 22, 2021, the disclosure of which is incorporated by reference herein in its entirety as part of the present application.

### REFERENCE TO SEQUENCE LISTING

A computer readable XML file entitled “GWP20220801353-sequence listing.xml”, that was created on Dec. 16, 2022, with a file size of about 16,936 bytes, contains the sequence listing for this application, has been filed with this application, and is hereby incorporated by reference in its entirety.

### TECHNICAL FIELD

The present disclosure relates to the field of biotechnology, in particular to a biomarker, a kit and a method for detection or auxiliary detection of proton irradiation, and use thereof.

### BACKGROUND ART

During the implementation of projects such as spacecraft interactive docking, exit activities, establishment of space stations, long-term manned space missions, and manned moon landings, astronauts in spacecraft cabins, especially outside the cabin, will face health hazards from exposure to complex space radiation environments. Although the space radiation environment is extremely complex, protons are still the main component of space radiation and play an important role in space radiation.

Proton radiation can ionize the molecules or atoms of the affected substance. When it acts on the human body or organism, it can cause changes in the atoms or molecules in tissue cells, causing the irradiated cells to be killed or mutated, thereby resulting in various hazardous effects on human health. However, there is a lack of simple and rapid methods for detecting proton irradiation in the prior art. Therefore, it is ever urgent to develop a rapid and safe detection technology to monitor the exposure dose on astronauts.

### SUMMARY

An objective of the present disclosure is to provide a microbial biomarker for detection or auxiliary detection of proton irradiation. Whether a subject to be tested is exposed to proton radiation is accurately determined by detection of an abundance or relative abundance of DNA of the microbial biomarker.

In order to solve the above-mentioned technical problems, the present disclosure provides the following technical solutions.

The present disclosure provides a microbiota as a biomarker for determination or auxiliary determination of whether a sample is exposed to proton radiation, in which the microbiota includes at least one of Cyanobacteria and Epsilonbacteraeota, Anaerovorax and Helicobacter.

The present disclosure provides use of the microbiota as a biomarker in the preparation of a product for determination or auxiliary determination of whether a sample is exposed to proton radiation.

In some embodiments, whether a sample is exposed to proton radiation is determined by detecting an abundance or relative abundance of microbiota DNA in the sample.

In a further embodiment, the sample is feces.

The present disclosure provides a kit, including a reagent for detecting an abundance or relative abundance of the microbiota DNA.

In some embodiments, the reagent includes a primer or a probe for detecting the abundance or relative abundance of the microbiota DNA.

In a further embodiment, the primer is selected from the group consisting of primer set 1, primer set 2, primer set 3 and primer set 4;

In the primer set 1, an upstream primer has the sequence set forth in SEQ ID NO.1, and a downstream primer has the sequence set forth in SEQ ID NO.2;

In the primer set 2, an upstream primer has the sequence set forth in SEQ ID NO.3, and a downstream primer has the sequence set forth in SEQ ID NO.4;

In the primer set 3, an upstream primer has the sequence set forth in SEQ ID NO.5, and a downstream primer has the sequence set forth in SEQ ID NO.6;

In the primer set 4, an upstream primer has the sequence set forth in SEQ ID NO.7, and a downstream primer has the sequence set forth in SEQ ID NO.8.

The present disclosure also provides use of the kit in determination or auxiliary determination of whether a sample is exposed to proton radiation.

The present disclosure also provides a method for determination or auxiliary determination of whether a sample is exposed to proton radiation, including:

Extracting total microbiota DNA in the sample for Polymerase chain Reaction (PCR) amplification, subjecting a resulting PCR amplification product to sequencing and data analysis to obtain an abundance or relative abundance of the microbiota DNA, and determining whether the sample is exposed to proton radiation.

In some embodiments, the abundance or relative abundance of the microbiota DNA in a sample irradiated by protons is greater than those of normal microbiota DNA.

The disclosure provides a microbial biomarker for detection or auxiliary detection of proton irradiation. After Balb/c mice and C57BL/6 mice are irradiated, an increase in abundances of Cyanobacteria, Epsilonbacteraeota, Anaerovorax and Helicobacter is detected. Whether a subject to be tested is exposed to proton radiation can be determined by comparing the abundances or relative abundances of Cyanobacteria, Epsilonbacteraeota, Anaerovorax, Helicobacter with those in Balb/c mice and C57BL/6 mice that are not exposed to proton radiation. This is conductive to timely accident rescue, casualty treatment and clinical treatment application, which is of great significance to radiation treatment.

### BRIEF DESCRIPTION OF THE DRAWINGS

FIG. 1 shows a plot representing Principal Co-ordinates Analysis (PCoA) of feces microbiota collected from Balb/c mice and C57BL/6 mice with or without proton irradiation.

FIG. 2 shows results of the composition analysis of microbiota in feces collected from Balb/c mice and C57BL/6 mice with or without proton irradiation at phylum-level.

FIG. 3A-3B are analysis of the significant differences in the bacterial phylum of feces collected from Balb/c mice and C57BL/6 mice with or without proton irradiation; in which A shows phyla with changed abundance in Balb/c mice after proton irradiation, and B shows phyla with increased abundance in C57BL/6 mice after proton irradiation.

FIG. 4A-4B are analysis of the significant differences of microbiota in feces collected from Balb/c mice and C57BL/6 mice with or without proton irradiation at genus level; in which A shows genera with changed abundance in Balb/c mice after proton irradiation and B shows genera with changed abundance in C57BL/6 mice after proton irradiation.

### DETAILED DESCRIPTION OF THE EMBODIMENTS

The present disclosure provides a microbiota as a biomarker for detection or auxiliary detection of proton irradiation, and the microbiota includes at least one of Cyanobacteria, Epsilonbacteraeota, Anaerovorax, and Helicobacter. In the present disclosure, the abundance or relative abundance of the DNA of Cyanobacteria, Epsilonbacteraeota, Anaerovorax, and Helicobacter is increased in the sample subjected to proton radiation.

The present disclosure provides use of the microbiota as a biomarker in the preparation of a product for determination or auxiliary determination of whether a sample is exposed to proton radiation. In the present disclosure, whether a sample is exposed to proton radiation is determined by detecting the abundance or relative abundance of microbiota DNA in the sample. In some embodiments, the sample is feaces from the sample to be tested. In a specific embodiment of the present disclosure, the abundance or relative abundance of the microbiota DNA is preferably those in the colorectal feces of C57BL/6 mice and Balb/c mice.

The present disclosure provides a kit, including a reagent for detecting an abundance or relative abundance of microbiota DNA. In some embodiments of the present disclosure, the reagent includes a primer or a probe for detecting the abundance or relative abundance of the microbiota DNA. The primer is selected from the group consisting of primer set 1, primer set 2, primer set 3 and primer set 4; in the primer set 1, an upstream primer has the sequence set forth in SEQ ID NO.1, and a downstream primer has the sequence set forth in SEQ ID NO.2; in the primer set 2, an upstream primer has the sequence set forth in SEQ ID NO.3, and a downstream primer has the sequence set forth in SEQ ID NO.4; in the primer set 3, an upstream primer has the sequence set forth in SEQ ID NO. 5, and a downstream primer has the sequence set forth in SEQ ID NO.6; in the primer set 4, an upstream primer has the sequence set forth in SEQ ID NO.7, and a downstream primer has the sequence set forth in SEQ ID NO.8. In the present disclosure, by amplification of different regions of the microbiota DNA using the primers, the gene abundance or relative abundance of the region is detected. The gene fragments amplified by the primers are shown in Table 1 below:

TABLE 1
Sequences of the primer sets

| | Amplification | | |
|---|---|---|---|
| Primer | region | Primer sequence | SEQ ID NO. |
| Primer | V3-V4 | 341F (5′ -CCTACGGGNGGCWGCAG-3′) | SEQ ID NO. 1 |
| set 1 | | 805R(5′-GACTACHVGGGTATCTAATCC-3′) | SEQ ID NO. 2 |
| Primer | Archaeobacteria | F(5′-GYGCASCAGKCGMGAAW-3′) | SEQ ID NO. 3 |
| set 2 | | R(5′-GGACTACHVGGGTWTCTAAT-3′) | SEQ ID NO. 4 |
| Primer | V4 | 515F(5′-GTGYCAGCMGCCGCGGTAA-3′) | SEQ ID NO. 5 |
| set 3 | | 806R (5′ GGACTACHVGGGTWTCTAAT-3′) | SEQ ID NO. 6 |
| Primer | V4-V5 | F(5′-GTGCCAGCMGCCGCGG-3′) | SEQ ID NO. 7 |
| set 4 | | R(5′-CCGTCAATTCMTTTRAGTTT-3′) | SEQ ID NO. 8 |

The present disclosure further provides use of the kit in the detection or auxiliary detection of whether a sample is exposed to proton radiation. In some embodiments of the present disclosure, the use is to amplify the microbiota DNA with the kit to determine the abundance or relative abundance of the microbiota DNA, so as to determine whether the sample to be tested is exposed to proton radiation.

The present disclosure further provides a method for detection or auxiliary detection of proton irradiation, including:

Extracting total microbiota DNA in a sample for PCR amplification, subjecting an obtained PCR amplification product to sequencing and data analysis to obtain an abundance or relative abundance of the microbiota DNA to determine whether the sample is exposed to proton radiation.

In some embodiments of the present disclosure, after extraction of the total microbiota DNA, the quality of the extracted DNA is detected using a method of agarose gel electrophoresis. In some embodiments of the present disclosure, after quality detection of the extracted DNA, quantification is performed; the method of quantification is preferably ultraviolet spectrophotometer-based method. The method for extracting the total microbiota DNA in the sample in the present disclosure is not specifically limited. In a specific embodiment of the present disclosure, the method is the CTAB method. In the present disclosure, the sequencing platform is not specifically limited. In a specific embodiment of the present disclosure, the sequencing platform is preferably a NovaSeq 6000 sequencer.

In the present disclosure, the data analysis can be used to convert the abundance or relative abundance of the microbiota DNA from the sample to be tested into the detection result of the sample to be tested. In the present disclosure, the data analysis includes a data processing module, a diversity analysis module, species annotation and differential analysis, as well as an advanced analysis module; in which, the data processing module is used to split the paired-end data obtained by sequencing, remove the linker and barcode sequence, then assembly and filter the data, and perform denoising using Divisive Amplicon Denoising Algorithm 2 (DADA2); the diversity analysis module is used to compare the abundance or relative abundance of the microbiota DNA from the object to be tested with that from an object that is not exposed to proton radiation; species annotation, differential analysis and advanced analysis modules are used to output conclusions. In the present disclosure, the software and versions thereof used in the data analysis are shown in Table 2.

TABLE 2
Software version and analysis requirements

| Analysis | Software | Version |
|---|---|---|
| Sequence | FLASH(http://ccb.jhu.edu/software/FLASH/) | v1.2.8 |
| assembly | | |
| Filtering of low | fqtrim(http://ccb.jhu.edu/software/fqtrim/) | v0.94 |
| quality reads | | |
| Filtering of | Vsearch(https://github.com/torognes/vsearch) | v2.3.4 |
| chimera | | |
| Noise reduction | qiime2(https://qiime2.org/) | 2019.7 |
| (DADA2) | | |
| Removal of | cutadapt(http://cutadapt.readthedocs.org/en/ | v1.9 |
| sequencing | stable/guide.html) | |
| adapters | | |
| Removal of low | fqtrim(http://ccb.jhu.edu/software/fqtrim/) | v0.94 |
| quality reads | | |
| PCoA | Omicstudio tools | V8.4.0 |
| | (https://www.omicstudio.cn/tool/24 ) | |
| Bar plot | Omicstudio tools | V8.4.0 |
| | (https://www.omicstudio.cn/tool/17) | |
| Boxplot | Omicstudio tools | V8.4.0 |
| | (https://www.omicstudio.cn/tool/1) | |

In the present disclosure, if the abundance or relative abundance of the microbiota DNA from the sample to be tested is greater than that from the sample that is not exposed to proton radiation, it is determined that the sample to be tested is exposed to proton radiation; if the abundance or relative abundance of the microbiota DNA of the sample to be tested is less than or equal to that from the sample that is not exposed to proton radiation, it is determined that the sample to be tested is not exposed to proton radiation.

In the present disclosure, the raw materials, reagents and equipment used are all known products, and conventional commercially available products can be used.

In order to further illustrate the present disclosure, the technical solutions provided by the present disclosure are described in detail below with reference to the examples, but they should not be construed as limiting the protection scope of the present disclosure.

### Example 1

A method for determining whether a sample is exposed to proton radiation using at least one of the microbiota biomarkers Cyanobacteria, Epsilonbacteraeota, Anaerovorax, and Helicobacter:

1. Experimental Procedure

1.1 Extraction of Total Microbiota DNA

The total microbiota DNA was extracted from mouse fecal samples by CTAB method, the quality of the extracted DNA was detected by agarose gel electrophoresis, and the DNA was quantified by UV spectrophotometer.

1.2 PCR Amplification

The DNA obtained in step 1.1 was amplified with the primer set in Table 1, in which the system and conditions of the PCR reaction are shown in Table 3 and Table 4, respectively.

TABLE 3
PCR reaction system

| Component | Volume |
|---|---|
| Phusion Hot start flex 2X Master Mix | 12.5 μl |
| Forward Primer | 2.5 μl |
| Reverse Primer | 2.5 μl |
| Template DNA | 50 ng |
| Add ddH₂O to | 25 μl |

TABLE 4
PCR reaction conditions

| Temperature | Time | Cycle |
|---|---|---|
| 98° C. | 30 seconds | |
| 98° C. | 10 seconds | |
| 54° C. | 30 seconds | 35 |
| 72° C. | 45 seconds | |
| 72° C. | 10 minutes | |
| 4° C. | ∞ | |

1.3 Quantification of PCR Product

The PCR products obtained in step 1.2 were purified with AMPure XT beads (Beckman Coulter Genomics, Danvers, Mass., USA), then quantified by Qubit (Invitrogen, USA), and then mixed in the corresponding proportions according to the sequencing volume requirements of the samples.

1.4 Recovery and Purification of Amplification Products

PCR amplification products were detected by 2% agarose gel electrophoresis, and recovered using AMPure XT beads recovery kit.

1.5 Quantification, Pooling and Sequencing of Amplified Products

The purified PCR products were evaluated using Agilent 2100 Bioanalyzer (Agilent, USA) and the library quantification kit from Illumina (Kapa Biosciences, Woburn, Mass., USA). The concentration of qualified library should be above 2 nM. The qualified on-board sequencing libraries (Index sequence not repeated) were each serially diluted, mixed according to the required sequencing amount at the corresponding ratio, and denatured into single-stranded by NaOH for paired-end sequencing (2×250 bp) on a NovaSeq 6000 sequencer using the NovaSeq 6000 SP Reagent Kit (500 cycles).

### Example 2

Bioinformatics Analysis

1. Data De-Multiplexing

For the paired-end data obtained by sequencing, it is necessary to de-multiplex the sample data according to the barcode information, and remove the adapter and barcode sequences.

2. Data Registration and Filtering

The specific steps were as follows:

1) The primer sequence and sequences containing bases to reduce bias in RawData were removed; in which, the software used was cutadapt (v1.9), and the parameters were as follows: ‘-g R1 -G R2 -n 1 -O 17 -m 100’;

2) Each pair of paired-end reads were assembled into a longer tag according to the overlap area; the software used is FLASH (v1.2.8), and the parameters were as follows: ‘-m 10 -M 100 -x 0.25 -t 1 -z’;

3) The quality of the sequencing reads were scanned by the slip window method, with a default window size being 100 bp; when the average quality in the window was lower than 20, the part of the read from the start of the window to the 3′ end was cut off; in which the software used was fqtrim, and the parameters were as follows: ‘-P 33 -w 100 -q 20-1100 -m 5 -p 1 -V -o trim.fastq.gz’;

4) the truncated sequences with length less than 100 bp were removed;

5) sequences with a truncated N (undetermined ambiguous base) content of more than 5% were removed;

6) the chimera sequences were removed; in which, the software used was Vsearch (v2.3.4), and the parameters were default;

3. DADA2 Denoising

DADA2 was called through qiime dada2 denoise-paired option to perform length filtering and denoising. Amplicon sequence variant (ASV) (feature) sequences and ASV (feature) abundance tables were obtained, and singletons ASVs were discarded (that is, ASVs (feature) that appear only once (the total number of sequence=1) in the entire samples, using default parameters).

4. Diversity Analysis

Alpha diversity analysis and beta diversity analysis were performed based on the obtained ASV (feature) sequences and ASV (feature) abundance tables. In the analysis, the alpha diversity analysis mainly evaluated the diversity within the habitat through the seven indexes of observed species, shannon, simpson, chao1, goods_coverage and pielou_e. Beta diversity analysis mainly evaluated and analyzes the diversity between habitats (samples/groups) by calculating four distances (weighted_unifrac, unweighted_unifrac, jaccard, bray_curtis).

5. Species Annotation

According to the ASV (feature) sequence file, the SILVA (Release 138, https://www.arbsilva.de/documentation/release138/) database and NT-16S database were used for species annotation, and the abundance of each species was counted according to the ASV (feature) abundance table. The confidence threshold for annotation was 0.7.

6. Difference Analysis and Advanced Analysis

Based on the obtained species abundance statistics, a difference analysis between each comparison group was performed. Different statistical methods were selected according to the sample conditions: Fisher's exact test was used to compare the differences between samples without biological replicates; Mann-Whitney U test was used to compare the differences between two groups of samples with biological replicates; and Kruskal-Wallis test was used to compare the differences among multiple groups of samples with biological replicates, with a screening threshold of p<0.05.

### Example 3

1. 6-8 week-old male mice were raised 1 week in a room for Specific Pathogen Free (SPF) animal before proton irradiation for adaption to the environment. The mice were provided with sufficient water and feed, and kept in a 12 hours light:12 hours dark cycle. The experiments were approved by the animal ethics committee of the Institute of Radiation Medicine of the Academy of Military Medical Sciences. The mice were Balb/c mice and C57BL/6 mice, which were purchased from SPF biotechnology co., LTD.

The mice were placed in the device and subjected to 5Gy whole body irradiation in sequence. After 3 days, about 5 mouse feces were taken under aseptic conditions and placed in a cryopreservation tube, immediately frozen with liquid nitrogen for 1 hour, and stored at −80° C. until use. The proton radiation source was from the 90 MeV single-energy protons generated by the 100 MeV proton cyclotron of the China Institute of Atomic Energy.

2. The abundance or relative abundance of the DNA from mice before and after proton irradiation was detected with the method described in Example 1.

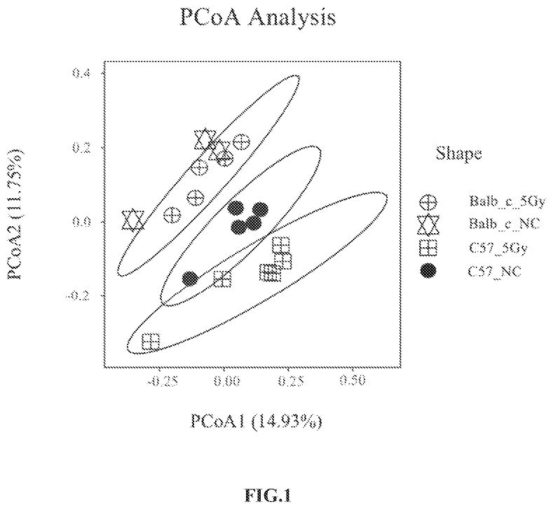

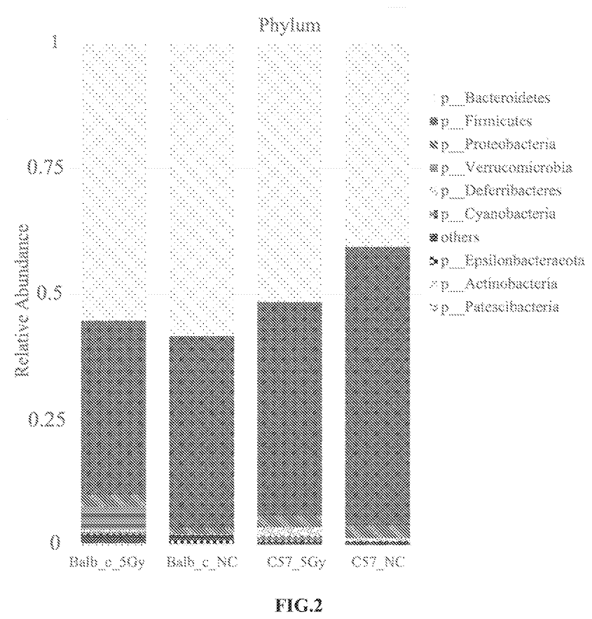

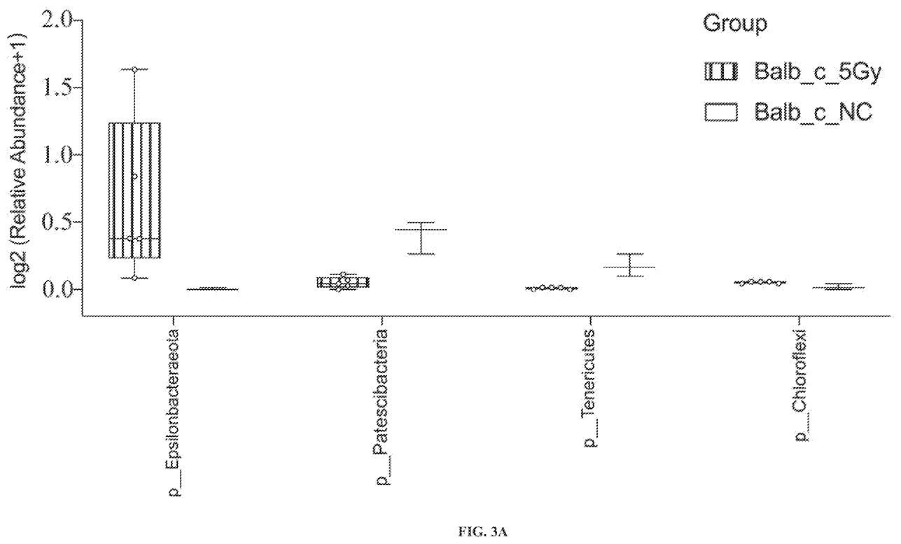

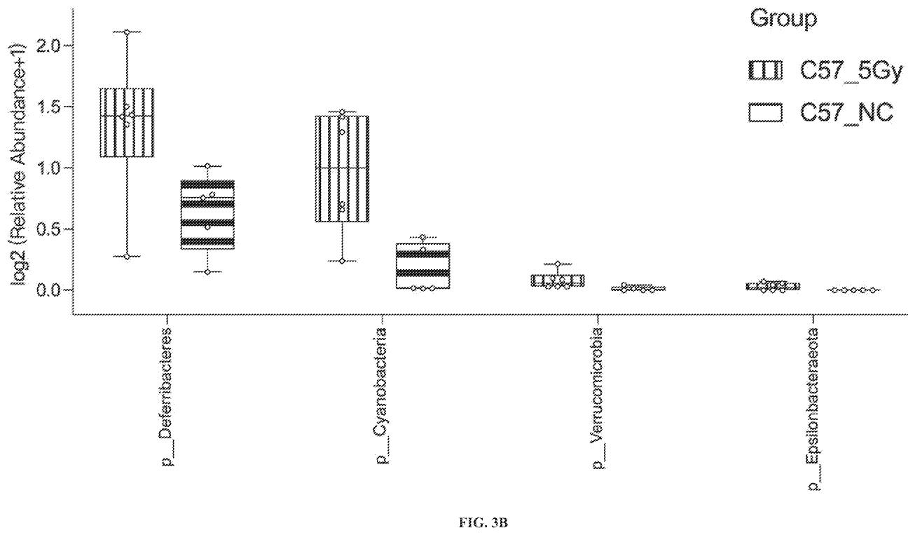

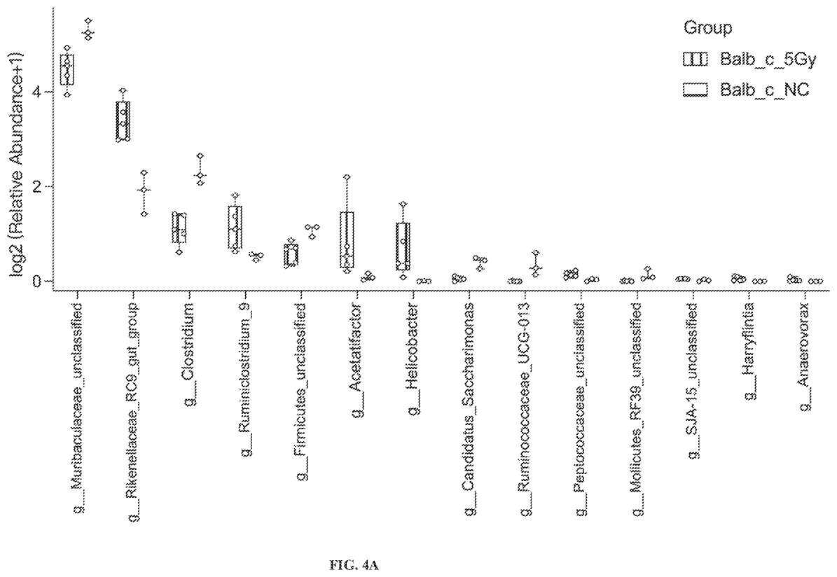

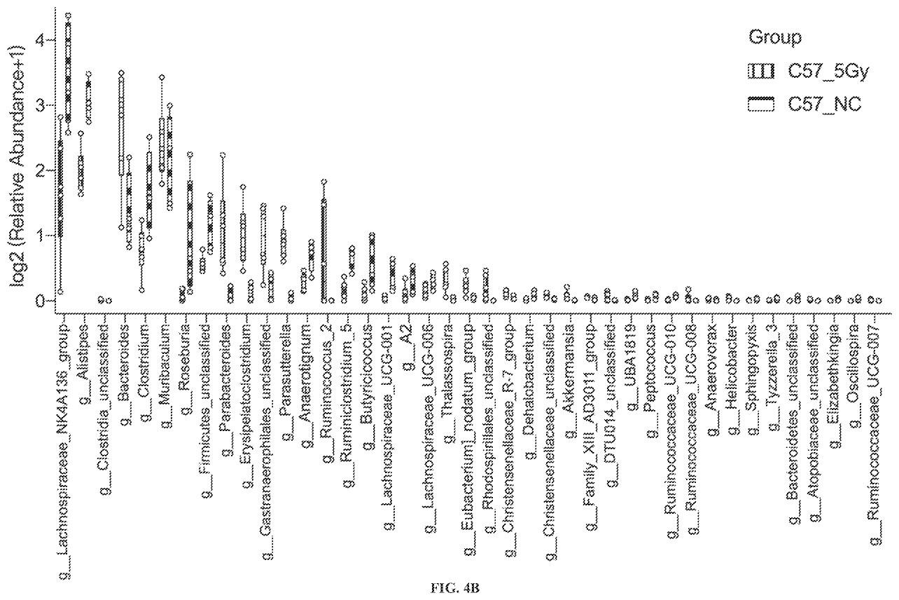

3. FIG. 1 was plotted using the omicstudio tools (https://www.omstudio.cn/tool/24) (version V8.4.0), FIG. 2 was plotted using the omicstudio tools (https://www.omstudio.cn/tool/17) (version V8.4.0), and FIG. 3-4 were plotted using the omicstudio tools (https://www.omstudio.cn/tool/1) (version V8.4.0).

As could be seen in FIG. 1, there was an obvious separation between the microbiota of C57BL/6 mice with and without proton irradiation, proving a significant difference and discrimination between the Balb/c mice with and without proton irradiation. The top ten changed flora in FIG. 2 were Bacteroidetes, Firmicutes, Proteobacteria, Verrucomicrobia, Deferribacteres, Cyanobacteria, Unclassified, Epsilonbacteraeota, Actinobacteria and Patescibacteria.

FIGS. 3A-3B and 4A-4B showed that Cyanobacteria and Epsilonbacteraeota all increased in the gut microbiota of Balb/c and C57BL/6 mice. Anaerovorax and genus Helicobacter of phylum Proteobacteria increased in the gut microbiota of Balb/c and C57BL/6 mice, while genus Clostridium of phylum Firmicutes and Firmicutes_unclassified decreased in both Balb/c and C57BL/6 mice.

As can be seen from the above examples, the abundance or relative abundance of the DNA of microbiota biomarkers Cyanobacteria, Epsilonbacteraeota, Anaerovorax, and Helicobacter from the mouse sample that is suffered from proton irradiation is greater than that from the mouse sample that is not exposed to proton radiation. It is indicated that the microbiota Cyanobacteria, Epsilonbacteraeota, Anaerovorax and Helicobacter can be used as biomarkers for determination or auxiliary determination of whether samples are exposed to proton radiation, which is of great significance to radiation treatment.

The above is only examples of the present disclosure, and is not intended to limit the scope of the present disclosure. Every equivalent structure or equivalent procedure generated by transformation according to the description of the present disclosure, or embodiments that are directly or indirectly used in other related fields are similarly included in the protection scope of the present disclosure.

```
Sequence Listing Information:
    DTD Version: V1_3
    File Name: GWP20220801353-sequence listing.xml
    Software Name: WIPO Sequence
    Software Version: 2.2.0
    Production Date: 2022-12-16
General Information:
    Current application / Applicant file reference: GWP20220801353
    Earliest priority application / IP Office: CN
    Earliest priority application / Application number: 202111575146.5
    Earliest priority application / Filing date: 2021-12-22
    Applicant name: Academy of Military Medical Sciences
    Applicant name / Language: en
    Invention title: BIOMARKER, KIT, AND METHOD FOR DETECTION OR
AUXILIARY DETECTION OF PROTON IRRADIATION AND USE THEREOF ( en )
    Sequence Total Quantity: 8
Sequences:
    Sequence Number (ID): 1
    Length: 17
    Molecule Type: DNA
    Features Location/Qualifiers:
      - source, 1..17
        > mol_type, other DNA
        > note, Primer 341F
        > organism, synthetic construct
      - misc_difference, 9
        > note, n is a, t, c or g
      - misc_difference, 13
        > note, w is a or t
    Residues:
    cctacgggng gcwgcag
17
    Sequence Number (ID): 2
    Length: 21
    Molecule Type: DNA
    Features Location/Qualifiers:
      - source, 1..21
        > mol_type, other DNA
        > note, Primer 805R
        > organism, synthetic construct
      - misc_difference, 7
        > note, h is a, t or c
      - misc_difference, 8
        > note, v is g, a or c
    Residues:
    gactachvgg gtatctaatc c
21
    Sequence Number (ID): 3
    Length: 17
    Molecule Type: DNA
    Features Location/Qualifiers:
      - source, 1..17
        > mol_type, other DNA
        > note, Forward primer of primer set 2
        > organism, synthetic construct
      - misc_difference, 2
        > note, y is c or t
      - misc_difference, 6
        > note, s is g or c
      - misc_difference, 10
        > note, k is g or t
      - misc_difference, 13
        > note, m is a or c
      - misc_difference, 17
        > note, w is a ort
    Residues:
    gygcascagk cgmgaaw
17
    Sequence Number (ID): 4
    Length: 20
    Molecule Type: DNA
    Features Location/Qualifiers:
      - source, 1..20
        > mol_type, other DNA
        > note, Reverse primer of primer set 2
        > organism, synthetic construct
      - misc_difference, 8
        > note, h is a, tor c
      - misc_difference, 9
        > note, v is g, a or c
      - misc_difference, 14
        > note, w is a or t
    Residues:
    ggactachvg ggtwtctaat
20
    Sequence Number (ID): 5
    Length: 19
    Molecule Type: DNA
    Features Location/Qualifiers:
      - source, 1..19
        > mol_type, other DNA
        > note, Primer 515F
        > organism, synthetic construct
      - misc_difference, 4
        > note, y is c or t
      - misc_difference, 9
        > note, m is a or c
    Residues:
    gtgycagcmg ccgcggtaa
19
    Sequence Number (ID): 6
    Length: 20
    Molecule Type: DNA
    Features Location/Qualifiers:
      - source, 1..20
        > mol_type, other DNA
        > note, Primer 806R
        > organism, synthetic construct
      - misc_difference, 8
        > note, h is a, t or c
      - misc_difference, 9
        > note, v is g, a or c
      - misc_difference, 14
        > note, w is a or t
      Residues:
      ggactachvg ggtwtctaat
20
    Sequence Number (ID): 7
    Length: 16
    Molecule Type: DNA
    Features Location/Qualifiers:
      - source, 1..16
        > mol_type, other DNA
        > note, Forward primer of primer set 4
        > organism, synthetic construct
      - misc_difference, 9
        > note, m is a or c
    Residues:
    gtgccagcmg ccgcgg
16
    Sequence Number (ID): 8
    Length: 20
    Molecule Type: DNA
    Features Location/Qualifiers:
      - source, 1..20
        > mol_type, other DNA
        > note, Reverse primer of primer set 4
        > organism, synthetic construct
      - misc_difference, 11
        > note, m is a or c
      - misc_difference, 15
        > note, r is a or g
    Residues:
    ccgtcaattc mtttragttt
20
END
```

1. A microbial biomarker for detection or auxiliary detection of proton irradiation, comprising at least one of Cyanobacteria, Epsilonbacteraeota, Anaerovorax, and Helicobacter.

2. (canceled)

3. (canceled)

4. (canceled)

5. A kit comprising a reagent for detecting an abundance or relative abundance of microbiota DNA.

6. The kit according to claim 5, wherein the reagent comprises a primer or a probe for detecting the abundance or relative abundance of the microbiota DNA.

7. The kit according to claim 6, wherein the primer is any one or several groups of primer set 1, primer set 2, primer set 3 and primer set 4;
in the primer set 1, an upstream primer has the sequence set forth in SEQ ID NO.1, and a downstream primer has the sequence set forth in SEQ ID NO.2;
in the primer set 2, an upstream primer has the sequence set forth in SEQ ID NO.3, and a downstream primer has the sequence set forth in SEQ ID NO.4;
in the primer set 3, an upstream primer has the sequence set forth in SEQ ID NO.5, and a downstream primer has the sequence set forth in SEQ ID NO.6;
in the primer set 4, an upstream primer has the sequence set forth in SEQ ID NO.7, and a downstream primer has the sequence set forth in SEQ ID NO.8.

8. (canceled)

9. A method for determination or auxiliary determination of whether a sample is exposed to proton radiation, comprising
extracting total DNA of at least one of Cyanobacteria, Epsilonbacteraeota, Anaerovorax, and Helicobacter in the sample for PCR amplification, subjecting a resulting PCR amplification product to sequencing and data analysis to obtain an abundance or relative abundance of microbiota DNA, and determining whether the sample is exposed to proton radiation.

10. The method according to claim 9, wherein the abundance or relative abundance of the microbiota DNA from the sample that is exposed proton radiation is greater than that of normal microbiota DNA.

11. The method according to claim 9, wherein the sample is feces.


SEQUENCE LISTING (8 sequences)

SEQ ID NO: 1 - DNA, 17 bp, synthetic construct
  source 1..17: mol_type = other DNA; note = Primer 341F
  misc_difference 9: note = n is a, t, c or g
  misc_difference 13: note = w is a or t
cctacgggnggcwgcag

SEQ ID NO: 2 - DNA, 21 bp, synthetic construct
  source 1..21: mol_type = other DNA; note = Primer 805R
  misc_difference 7: note = h is a, t or c
  misc_difference 8: note = v is g, a or c
gactachvgggtatctaatcc

SEQ ID NO: 3 - DNA, 17 bp, synthetic construct
  source 1..17: mol_type = other DNA; note = Forward primer of primer set 2
  misc_difference 2: note = y is c or t
  misc_difference 6: note = s is g or c
  misc_difference 10: note = k is g or t
  misc_difference 13: note = m is a or c
  misc_difference 17: note = w is a or t
gygcascagkcgmgaaw

SEQ ID NO: 4 - DNA, 20 bp, synthetic construct
  source 1..20: mol_type = other DNA; note = Reverse primer of primer set 2
  misc_difference 8: note = h is a, t or c
  misc_difference 9: note = v is g, a or c
  misc_difference 14: note = w is a or t
ggactachvgggtwtctaat

SEQ ID NO: 5 - DNA, 19 bp, synthetic construct
  source 1..19: mol_type = other DNA; note = Primer 515F
  misc_difference 4: note = y is c or t
  misc_difference 9: note = m is a or c
gtgycagcmgccgcggtaa

SEQ ID NO: 6 - DNA, 20 bp, synthetic construct
  source 1..20: mol_type = other DNA; note = Primer 806R
  misc_difference 8: note = h is a, t or c
  misc_difference 9: note = v is g, a or c
  misc_difference 14: note = w is a or t
ggactachvgggtwtctaat

SEQ ID NO: 7 - DNA, 16 bp, synthetic construct
  source 1..16: mol_type = other DNA; note = Forward primer of primer set 4
  misc_difference 9: note = m is a or c
gtgccagcmgccgcgg

SEQ ID NO: 8 - DNA, 20 bp, synthetic construct
  source 1..20: mol_type = other DNA; note = Reverse primer of primer set 4
  misc_difference 11: note = m is a or c
  misc_difference 15: note = r is a or g
ccgtcaattcmtttragttt